(19) United States
(12) Patent Application Publication
(10) Pub. No.: US 2026/0008751 A1
(43) Pub. Date: Jan. 8, 2026

(54) NITROXYL POLYPHENOL DERIVATIVE, METHODS FOR THE PREPARATION THEREOF AND USE THEREOF

(71) Applicants: UNIWERSYTET WARSZAWSKI, Warszawa (PL); INSTYTUT BIOLOGII DOSWIADCZALNEJ IM. M. NENCKIEGO PAN, Warszawa (PL)

(72) Inventors: Adrian Konopko, Warszawa (PL); Katarzyna Sektas, Warszawa (PL); Grzegorz Litwinienko, Warszawa (PL); Anna Bielak-Zmijewska, Warszawa (PL)

(73) Assignees: UNIWERSYTET WARSZAWSKI, Warszawa (PL); INSTYTUT BIOLOGII DOSWIADCZALNEJ IM. M. NENCKIEGO PAN, Warszawa (PL)

(21) Appl. No.: 18/881,181

(22) PCT Filed: Jul. 4, 2023
(86) PCT No.: PCT/PL2023/050049

§ 371 Date: Jan. 3, 2025

(30) Foreign Application Priority Data
Jul. 7, 2022 (PL) P.441664

(51) Int. Cl. C07D 211/94; A61K 31/452; A61K 31/4545; A61P 39/06; A61P 43/00

(52) U.S. Cl. CPC C07D 211/94; A61K 31/452; A61K 31/4545; A61P 39/06; A61P 43/00

(57) ABSTRACT

The object of the invention is a nitroxyl polyphenol derivative of formula:

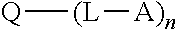

wherein Q is a group derived from a polyphenol;
L is an ester linking group containing 1 to 3 carbon atoms;
A is a 5- or 6-membered heterocyclic group containing one nitrogen atom which is in the form of nitroxyl radical (NO·), in which both carbon atoms adjacent to the nitroxyl radical are substituted independently of each other with one or two C₁-C₃ alkyl groups; and
n is an integer of 1 to 5.

The object of the invention is also methods for the preparation of the above-defined derivative and a use thereof as an antioxidant agent, in particular an antiaging agent.

The object of the invention is a nitroxyl polyphenol derivative, methods for the preparation thereof and a use thereof as an antioxidant agent, in particular an antiaging agent in cosmetology, pharmacy and medicine.

Cellular aging is the primary cellular process that occurs in adult organisms, and it is also necessary for normal organogenesis (embryonic development). Cellular aging is associated with all medical conditions defined as old age diseases (e.g. type 2 diabetes mellitus, atherosclerosis, neurodegenerative diseases) and involves increased inflammation in tissues and organs. The progress of civilization increases exposure to compounds that accelerate aging. In addition, methods used for therapeutic purposes (radio- and chemotherapy) cause more rapid aging of normal cells as a side effect. Aging is associated with excessive exposure to UV light (skin photoaging). Increased number of senile cells is observed after transplantation. Preventing or reducing cellular aging effects may be helpful for maintaining the health and good performance of the body for a longer time and may contribute to preventing and/or eliminating effects of diseases specific for the elderly age and undesirable effects of therapy.

Currently, novel compounds having potential antiaging activity are being continuously sought, and polyphenols are a highly promising class of compounds. However, the use of these compounds involves many limitations, such as limited bioavailability and the need for employing high concentrations to ensure effective action.

In connection with the above, it is justified to search for novel derivatives based on polyphenols, the use of which would not be associated with the aforementioned disadvantages.

Prior art describes derivatives of resveratrol and curcumin, which contain amino acids (e.g. phenylalanine, threonine, leucine or proline) added as the result of esterification reactions. (J. R. Manjunatha, B. K. Bettadaiah, P. S. Negi and P. Srinivas, Food Chemistry, Vol. 139, No. 1-4, 15 Aug. 2013, p. 332-338 and A. Mattarei, M. Azzolini, M. La Spina, M. Zoratti, C. Paradisi, L. Biasutto, Scientific Reports, 5, 14Oct. 20215, p. 1-11). However, no information about the antioxidant/antiaging activity of the aforementioned derivatives is found in the cited publications.

EP 2 189 155 A1 only generally discloses that compositions showing antiaging activity with respect to the human skin may contain a polyphenol as an antioxidant.

Therefore, the aim of the invention was to design and synthesize polyphenol derivatives showing antioxidant, and in particular antiaging, activity, that would solve the problem of low bioavailability and the need for employing high concentrations of the compounds.

The object of the invention is a nitroxyl polyphenol derivative of formula:

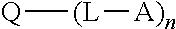

wherein Q is a group derived from a polyphenol;
L is an ester linking group containing 1 to 3 carbon atoms;
A is a 5- or 6-membered heterocyclic group containing one nitrogen atom which is in the form of nitroxyl radical (NO·), in which both carbon atoms adjacent to the nitroxyl radical are substituted independently of each other with one or two C₁-C₃ alkyl groups; and
n is an integer of 1 to 5.

Preferably, Q is a derivative of curcumin, quercetin, genistein or daidzein, and L is an ester linking group of formula —OC(O)—(CH₂)ₓ—, wherein x is an integer of 0 to 2, and more preferably, Q is a derivative of curcumin, and L is an ester linking group of formula —OC(O)—(CH₂)ₓ—, wherein x is an integer of 0 to 2.

Also preferably, Q is a derivative of resveratrol.

Preferably, L in a nitroxyl derivative of resveratrol is an ester linking group of formula —(CH₂)ₓ—C(O)O— or —OC(O)—(CH₂)ₓ—, wherein x is an integer of 0 to 2, and more preferably, L is an ester linking group of formula —(CH₂)ₓ—C(O)O—, wherein x is an integer of 0 to 2.

Preferably, A is a piperidine-1-oxyl group in which both carbon atoms adjacent to the nitroxyl radical are substituted independently of each other with two C₁-C₃ alkyl groups, and more preferably, A is 2,2,6,6-tetramethylpiperidine-1-oxyl group.

Preferably, n is 1 or 2.

Preferably, the aforementioned nitroxyl polyphenol derivative is the derivative of formula:

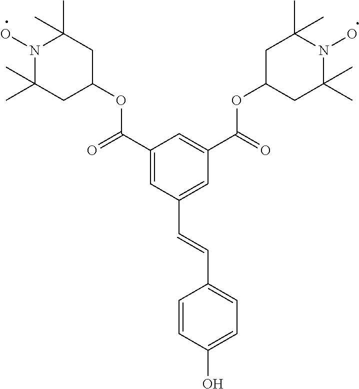

Preferably, the aforementioned nitroxyl polyphenol derivative is the derivative of formula:

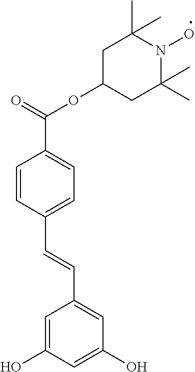

Preferably, the aforementioned nitroxyl polyphenol derivative is the derivative of formula:

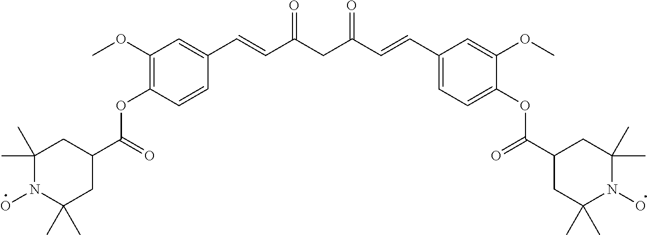

The object of the invention is a method for the preparation of nitroxyl polyphenol derivative of formula:

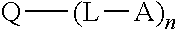

wherein Q is a group derived from a polyphenol;
L is an ester linking group of formula —OC(O)—(CH₂)ₓ—, wherein x is an integer of 0 to 2;
A is a 5- or 6-membered heterocyclic group containing one nitrogen atom which is in the form of nitroxyl radical (NO·), in which both carbon atoms adjacent to the nitroxyl radical are substituted independently of each other with one or two C₁-C₃ alkyl groups; and n is an integer of 1 to 5;
which comprises a reaction of a polyphenol with a 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with a carboxy group of general formula —(CH₂)ₓ—C(O)OH, wherein x is an integer of 0 to 2;
wherein the reaction is carried out in an organic solvent at a temperature in the range from −10 to 30° C., in the presence of a deprotonating agent for hydroxy group and an activating agent for carboxy group.

Preferably, the reaction is carried out for a time of 48 to 72 hours.

Preferably, a piperidine-1-oxyl compound in which both carbon atoms adjacent to the nitroxyl radical are substituted independently of each other with two C₁-C₃ alkyl groups and in which one of the other carbon atoms is substituted with a carboxy group of general formula —(CH₂)ₓ—C(O)OH, wherein x is an integer of 0 to 2, and more preferably, 4-carboxy-2,2,6,6-tetramethylpiperidine-1-oxyl compound, is used as the 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with a carboxy group of general formula —(CH₂)ₓ—C(O)OH, wherein x is an integer of 0 to 2.

Preferably, 4-dimethylaminopyridine is used as the deprotonating agent for hydroxy group.

Preferably, 1-ethyl-3-(3-dimethylaminopropyl)carbodiimide or N,N′-dicyclohexylcarbodiimide, and more preferably 1-ethyl-3-(3-dimethylaminopropyl)carbodiimide, is used as the activating agent for carboxy group.

Preferably, aprotic polar solvent, and more preferably dichloromethane, is used as the organic solvent.

Preferably, a polyphenol containing keto group, such as curcumin, quercetin, genistein or daidzein, and more preferably curcumin, is used as the polyphenol.

Preferably, when a polyphenol containing keto group is used, the reaction is carried out at a temperature in the range from −10° C. to a temperature lower than room temperature, and more preferably in the range from −10° C. to 0° C.

Preferably, resveratrol is used as the polyphenol.

Preferably, when resveratrol is used, the reaction is carried out at a temperature in the range from room temperature to 30° C.

The object of the invention is also a method for the preparation of nitroxyl polyphenol derivative of formula:

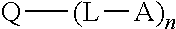

wherein:
Q is a derivative of resveratrol;
L is an ester linking group of formula —(CH₂)ₓ—C(O)O—, wherein x is an integer of 0 to 2;
A is a 5- or 6-membered heterocyclic group containing one nitrogen atom which is in the form of nitroxyl radical (NO·), in which both carbon atoms adjacent to the nitroxyl radical are substituted independently of each other with one or two C₁-C₃ alkyl groups; and
n is an integer of 1 or 2;
comprising the following steps:
a) a reaction between a benzaldehyde derivative substituted with at least one hydroxy group and a silylating agent to protect at least one hydroxy group, wherein the reaction is carried out at a temperature in the range from 0° C. to room temperature in an organic solvent in the presence of an activating agent for the silylating agent;
b) a reaction of the protected benzaldehyde derivative prepared in step a) with an alkyltriphenylphosphonium halide to convert aldehyde group to alkene group, wherein the reaction is carried out in an organic solvent at a temperature in the range from −78° C. to room temperature, in the presence of a strong base;
c) a coupling reaction of alkene group in the derivative prepared in step b) with a halogenated benzene derivative substituted with at least one ester group of formula —(CH₂)ₓ—C(O)OR, wherein R is a C₁-C₃ alkyl group, and x is an integer of 0 to 2; wherein the reaction is carried out in the presence of a catalyst, a phosphonium ligand and a base, optionally in an organic solvent, at a temperature in the range from room temperature to 190° C.;
d) a reaction of the derivative prepared in step c) with a reducing agent to reduce at least one ester group to at least one alcohol group, wherein the reaction is carried out in an organic solvent at a temperature in the range from −78° C. to room temperature;
e) a reaction of the derivative prepared in step d) with an oxidizing agent to oxidize at least one alcohol group to at least one aldehyde group, wherein the reaction is carried out in an organic solvent at a temperature in the range from 0° C. to room temperature;
f) a reaction of the derivative prepared in step e) with an oxidizing agent to oxidize at least one aldehyde group to at least one acid group, wherein the reaction is carried out in an organic solvent or a mixture of organic solvents at a temperature in the range from 0° C. to room temperature;
g) a reaction of the derivative prepared in step f) with a 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with hydroxy group, to prepare a nitroxyl polyphenol derivative containing at least one protected hydroxy group, wherein the reaction is carried out in an organic solvent at a temperature in the range from room temperature to 30° C., in the presence of a deprotonating agent for hydroxy group and an activating agent for carboxy group; and
h) a reaction of the nitroxyl polyphenol derivative containing at least one protected hydroxy group prepared in step g) with a reagent serving as a source of fluoride ions to deprotect at least one hydroxy group, wherein the reaction is carried out in an organic solvent at a temperature in the range from 0° C. to room temperature.

Preferably, in step a), hydroxy- or dihydroxybenzaldehyde is used as the benzaldehyde derivative.

Preferably, in step a), an alkylsilyl halide, and more preferably tert-butyldimethylsilyl chloride, is used as the silylating agent.

Preferably, in step a), imidazole, a mixture of triethylamine with 4-dimethylaminopyridine or with 1,8-diazabicyclo[5.4.0]undec-7-ene or a mixture of 18-crown-6 ether with potassium hydride, and more preferably imidazole, is used as the activating agent for the silylating agent.

Preferably, the reaction in step a) is carried out for a time of 4 to 24 hours.

Preferably, in step b), methyltriphenylphosphonium bromide is used as the alkyltriphenylphosphonium halide.

Preferably, the reaction in step b) is carried out for a time of 4 to 24 hours.

Preferably, in step b), n-butyllithium, lithium diisopropylamide, potassium tert-butoxide or potassium bis(trimethylsilyl)amide, and more preferably n-butyllithium, is used as the strong base.

Preferably, in step c), 4-iodobenzoic acid ethyl ester or dimethyl 5-bromoisophthalate is used as the halogenated benzene derivative substituted with at least one ester group of formula —(CH₂)ₓ—C(O)OR, wherein R is a C₁-C₃ alkyl group, and x is an integer of 0 to 2.

Preferably, in step c), a palladium (0) or (II) complex, and more preferably palladium acetate, is used as the catalyst.

Preferably, in step c), tri(o-tolyl)phosphine is used as the phosphonium ligand.

Preferably, in step c), triethylamine is used as the base.

Preferably, in step c), the organic solvent is the base being used.

Preferably, the reaction in step c) is carried out at a temperature in the range from 50 to 90° C.

Preferably, the reaction in step c) is carried out for a time of 20 to 40 hours.

Preferably, in step d), diisobutylaluminum hydride or lithium aluminum hydride, and more preferably diisobutylaluminum hydride, is used as the reducing agent.

Preferably, the reaction in step d) is carried out at a temperature in the range from −78° C. to −50° C.

Preferably, the reaction in step d) is carried out for a time of 1 to 24 hours.

Preferably, in step e), pyridinium dichromate, pyridinium chlorochromate, oxalyl chloride, triethylamine in dichloromethane, tetrapropylammonium perruthenate or 4-methylmorpholine 4-oxide in tetrahydrofuran, and more preferably pyridinium dichromate, is used as the oxidizing agent.

Preferably, the reaction in step e) is carried out for a time of 2 to 18 hours.

Preferably, the reaction in step e) is carried out at a temperature in the range from 10° C. to room temperature.

Preferably, in step f), NaClO₂ and NaH₂PO₄·H₂O solution is used as the oxidizing agent.

Preferably, in step f), tert-butanol, tetrahydrofuran or 2-methylbut-2-ene or a mixture thereof is used as the organic solvent.

Preferably, the reaction in step f) is carried out for a time of 2 to 8 hours.

Preferably, the reaction in step f) is carried out at a temperature in the range from 0 to 3° C.

Preferably, in step g), a hydroxypiperidine-1-oxyl compound, in which both carbon atoms adjacent to the nitroxyl radical are substituted independently of each other with two C₁-C₃ alkyl groups, and more preferably 4-hydroxy-2,2,6,6-tetramethylpiperidine-1-oxyl compound, is used as the 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with hydroxy group.

Preferably, in step g), 4-dimethylaminopyridine is used as the deprotonating agent for hydroxy group.

Preferably, in step g), 1-ethyl-3-(3-dimethylaminopropyl)carbodiimide or N,N′-dicyclohexylcarbodiimide, and more preferably 1-ethyl-3-(3-dimethylaminopropyl)carbodiimide, is used as the activating agent for carboxy group.

Preferably, the reaction in step g) is carried out for a time of 16 to 52 hours.

Preferably, in step h), tetra-n-butylamine fluoride is used as the reagent serving as a source of fluoride ions.

Preferably, the reaction in step h) is carried out for a time of 1 to 5 hours.

Preferably, in steps from a) to e) and g) and h), aprotic solvent, and more preferably dichloromethane, dimethylformamide, toluene, tetrahydrofuran, acetonitrile or ether, is used as the organic solvent.

The object of the invention is also a use of the aforementioned nitroxyl polyphenol derivative as an antioxidant agent, preferably as an antiaging agent.

The derivatives of the invention are a combination of two compounds, that is, a polyphenol and a compound containing the nitroxyl radical. It has been shown that the polyphenol alone and the compound containing the nitroxyl radical alone have some antioxidant activity. However, the resulting derivatives have much better activity than the components used as their building blocks. Owing to their antioxidant activity, the derivatives of the invention delay cellular aging caused by oxidizing agents that increase the level of reactive oxygen species. They show bioactive effects with respect to a number of molecular targets in the cell. The antioxidant properties of the derivatives of the invention can be modified by altering the parent molecule or the number of substituted/free hydroxy groups. For example, by leaving at least one free OH group in the resveratrol derivative, compounds active in a broad pH range are obtained, because the nitroxyl radical shows its effect in acidic pH, while the free OH group of resveratrol shows its effect in alkaline pH. Furthermore, the presence of keto moiety which can be converted into enol moiety (>C═O into ═C—OH) affects the antioxidant properties of derivatives of curcumin, quercetin, genistein and daidzein. Therefore, it is not crucial to leave the free OH group in these derivatives. In addition, the type of the group containing the nitroxyl radical being incorporated is also very important for antioxidant properties. Heterocyclic groups having substituents at the carbon atoms adjacent to the nitrogen atom in the form of nitroxyl radical, which increase the stability of the radical in cells and prevent its conversion to hydroxylamine, have been shown to be the most preferred.

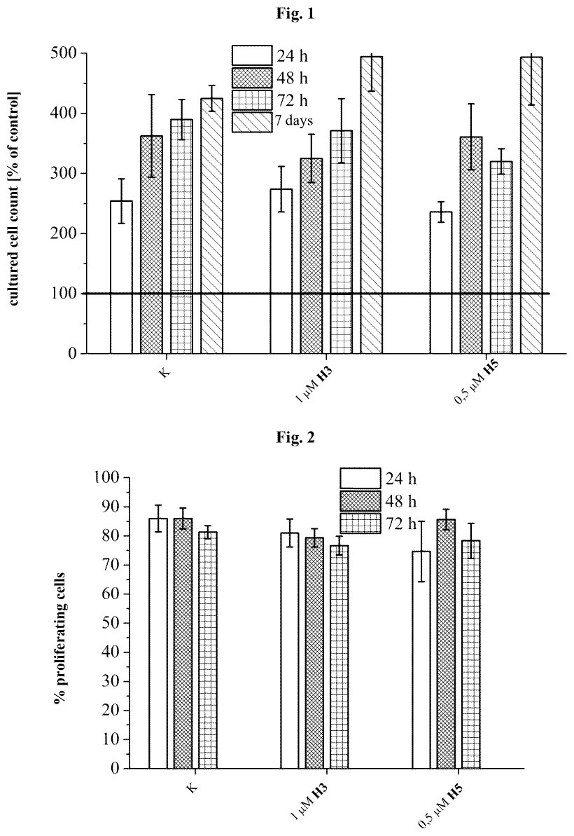

The results of studies on the antioxidant/antiaging properties of the derivatives of the invention with respect to the induced aging of human skin fibroblasts are shown in FIG. 1-6.

FIG. 1 illustrates the cytotoxic properties of the tested derivatives (H3 and H5) with respect to human skin fibroblasts.

FIG. 2 illustrates the effect of the tested derivatives (H3 and H5) on cell proliferation, determined based on the ability to incorporate bromodeoxyuridine (BrdU).

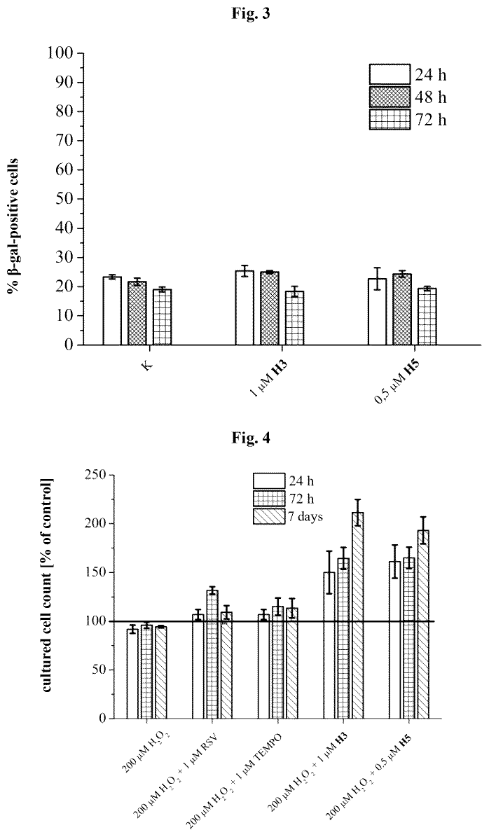

FIG. 3 illustrates the effect of the tested compounds (H3 and H5) on the activity of senescence-associated β-galactosidase (SA-β-gal) in cells.

FIG. 4-6 illustrate the protective properties of the tested compounds (H3 and H5) to prevent the effects of oxidative stress induced by hydrogen peroxide in cells.

The abbreviations and terms as used in the specification, in the Figures and in the claims have their commonly understood meanings as used by those skilled in the art to which this invention belongs. However, for clarity, the following terms and abbreviations should be understood as follows:

The term “a 5- or 6-membered heterocyclic group (heterocyclic compound) containing one nitrogen atom which is in the form of nitroxyl radical (NO·)” means a saturated, unsaturated or aromatic 5- or 6-membered heterocyclic group containing one nitrogen atom which is in the form of nitroxyl radical, such as piperidine-1-oxyl group, pyridine-1-oxyl group, pyrrolidine-1-oxyl group, pyrroline-1-oxyl group and pyrrole-1-oxyl group.

The term “a group derived from a polyphenol” means a polyphenol molecule, such as resveratrol, curcumin, quercetin, genistein and daidzein, in which one to all hydroxy groups may be substituted with the above-defined 5- or 6-membered heterocyclic group containing one nitrogen atom which is in the form of nitroxyl radical.

The term “an ester linking group containing from 1 to 3 carbon atoms” means groups of formula —(CH₂)ₓ—C(O)O— or —OC(O)—(CH₂)ₓ—, wherein x is an integer of 0 to 2, that is, groups, such as —(CH₂)₂—C(O)O—, —CH₂—C(O)O—, —C(O)O—, —OC(O)—(CH₂)₂—, —OC(O)—CH₂-lub-OC(O)—. The type of the ester linking group depends on the method for the preparation of nitroxyl polyphenol derivative.

The term “a C₁-C₃ alkyl group” means methyl, ethyl or propyl group.

The term “both carbon atoms adjacent to the nitroxyl radical are substituted independently of each other with one or two C₁-C₃ alkyl groups” means that each of the two carbon atoms adjacent to the nitroxyl radical may be substituted with a different or an identical number of C₁-C₃ alkyl groups, wherein the groups may be identical or different. Preferably, both carbon atoms are substituted with two C₁-C₃ alkyl groups.

Room temperature (rt) means a temperature in the range from 18 to 25° C.
THF: tetrahydrofuran
CH₂Cl₂: dichloromethane
4-carboxy-TEMPO: 4-carboxy-2,2,6,6-tetramethylpiperidine-1-oxyl compound
4-hydroxy-TEMPO: 4-hydroxy-2,2,6,6-tetramethylpiperidine-1-oxyl compound
TEMPO: 2,2,6,6-tetramethylpiperidine-1-oxyl compound
EDCl: 1-ethyl-3-(3-dimethylaminopropyl)carbodiimide
DMAP: 4-dimethylaminopyridine
Pd(OAc)₂: palladium acetate
DCC: N,N′-dicyclohexylcarbodimide
TBSCl: tert-butyldimethylsilyl chloride
CH₃PPh₃Br: methyltriphenylphosphonium bromide
Et₃N: triethylamine
ToP: tri(o-tolyl)phosphine
DIBALH: diisobutylaluminum hydride
TBAF: tetra-n-butylamine fluoride
PDC: pyridinium dichromate
t-BuOH: tert-butanol
nBuLi: n-butyllithium
EtOAc: ethyl acetate
Et₂O: diethyl ether
MeOH: methanol
CHCl₃: chloroform

The invention is illustrated by the following non-limiting examples. Unless specified otherwise, known reaction methods and commercially available equipment and reagents commonly used in the art to which this invention belongs, have been used in the examples which follow.

Example 1: The synthesis of nitroxyl polyphenol derivative in which all hydroxy groups have been substituted with the groups containing the nitroxyl radical.

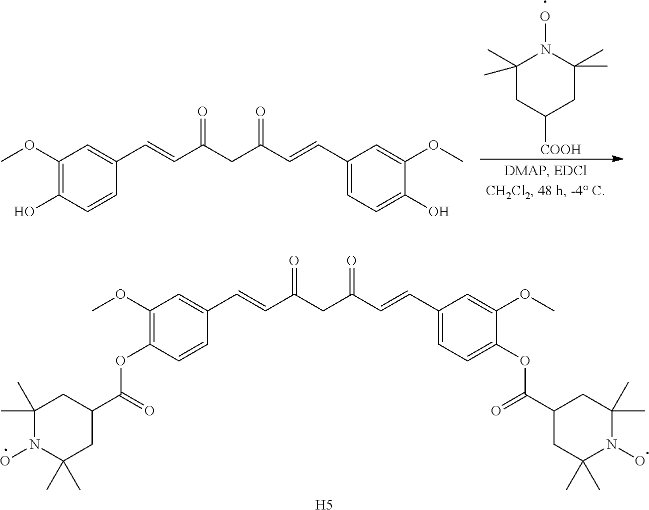

In a round-bottomed flask under argon atmosphere, curcumin (78.5 mg, 0.21 mmol), 4-carboxy-TEMPO (158.4 mg, 0.79 mmol), EDCI as the activating agent for carboxy group (86.1 mg, 0.55 mmol) and DMAP as the deprotonating agent for hydroxy group (30.3 mg, 0.25 mmol) were dissolved in 35 mL dichloromethane and stirred for 48 hours at −4° C. The reaction was also carried out using DCC instead of EDCI as the activating agent for carboxy group. After 2 days, the product was purified with column chromatography using CH₂Cl₂:MeOH (20:1) system as the eluent and silica gel as the stationary phase. In the subsequent step, the product was purified with preparative chromatography in which CHCl₃:MeOH (25:2) was used as the eluent. The resulting product was eluted from the silica gel using methanol and compound H5 in the form of orange solid was obtained (72.5 mg, 46%).

The synthesis of resveratrol derivative was carried out similarly.

In a round-bottomed flask under argon atmosphere, resveratrol (100 mg, 0.44 mmol), 4-carboxy-TEMPO (562.4 mg, 2.6 mmol), EDCI as the activating agent for carboxy group (238.1 mg, 1.53 mmol) and DMAP as the deprotonating agent for hydroxy group (26.8 mg, 0.22 mmol) were dissolved in 35 mL dichloromethane and stirred for 48 hours at room temperature. Subsequently, the product was purified using the method described above.

The aforementioned reactions are carried out in a temperature in the range from −10° C. to 30° C. However, the conducted experiments showed that in the case of polyphenols containing keto groups (—C(O)—) (curcumin, quercetin, genistein and daidzein), the reaction is preferably carried out at a temperature below room temperature (i.e. from −10° C. to a temperature below room temperature (about 17° C.)), and more preferably at a temperature in the range from −10° C. to 0° C. Whereas, in the case of polyphenols not containing keto groups (resveratrol), the aforementioned broader temperature range (i.e. from −10° C. to 30° C.) can be used without any problem, and the preferable range is from room temperature to 30° C. considering the more rapid progress of the reaction. Differences in the temperatures being used result from keto-enol equilibrium. Keto-enol equilibrium is shifted toward the enol form at room temperature, which may lead to the esterification of the enol group and substitution of the nitroxyl radical at this position. As the result, a mixture of different products can be obtained. Therefore, in the case of polyphenols containing keto groups, it is important to use lower temperature at which keto-enol equilibrium is not shifted toward the enol form.

Regarding the reaction time, extending it to 72 hours increases the yield of the reaction.

In the aforementioned syntheses, 4-carboxy-2,2,6,6-tetramethylpiperidine-1-oxyl compound was used as the heterocyclic compound containing one nitrogen atom in the form of nitroxyl radical. However, other compounds containing the nitroxyl radical being derivatives of piperidine, pyridine, pyrrolidine, pyrroline and pyrrole, in which both carbon atoms adjacent to the nitrogen atom in the form of the nitroxyl radical are substituted independently of each other with one or two (identical or different) methyl, ethyl or propyl groups, and in which one of the other carbon atoms is substituted with a carboxy group of formula —C(O)OH, —CH₂C(O)OH or —(CH₂)₂C(O)OH, can be also used in the identical conditions.

Any polar aprotic solvent can be used in the aforementioned reactions.

Using the above-defined method with minor modifications within the knowledge of one skilled in the art, a derivative of any polyphenol can be obtained in which all hydroxy groups are substituted with groups containing the nitroxyl radical. As described above, when selecting reaction parameters, attention should be paid to whether there are any keto groups in the polyphenol.

Example 2: The synthesis of nitroxyl resveratrol derivative containing at least one free hydroxy group.
a) Synthesis of the nitroxyl resveratrol derivative containing two free hydroxy groups:

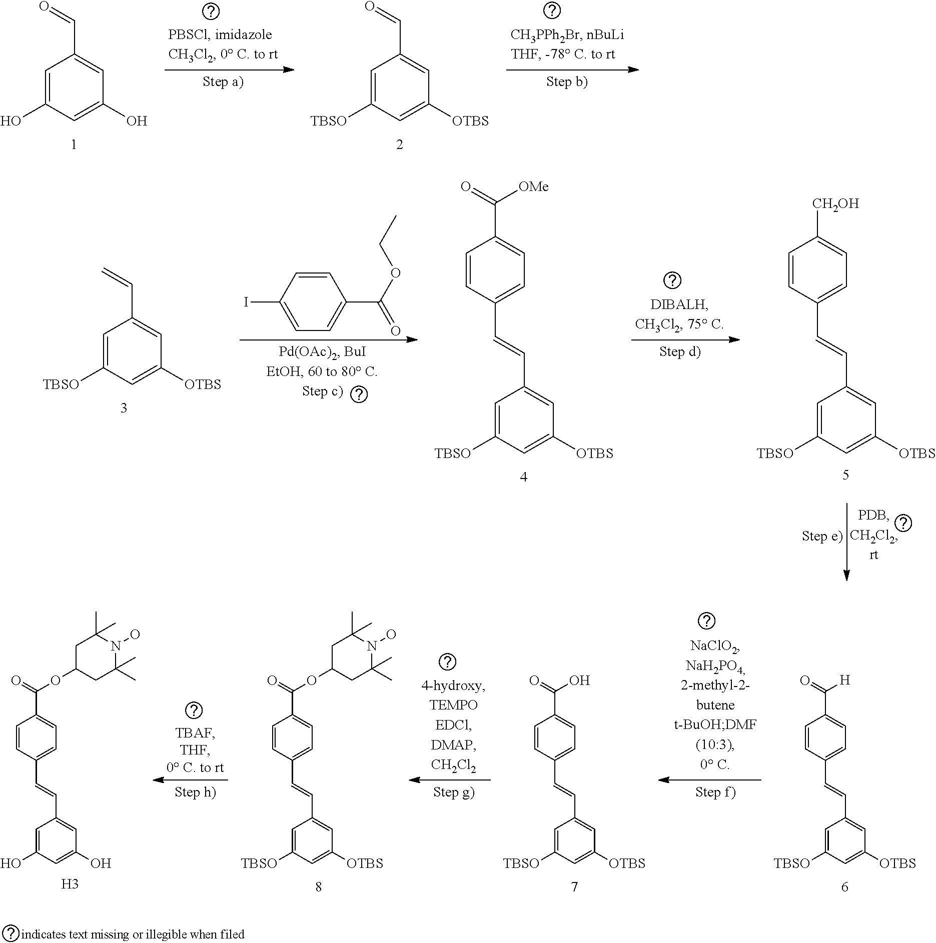

Step a): Imidazole as the activating agent for the silylating agent (5.923 g, 86.88 mmol) was added to the stirred suspension of 3,5-dihydroxybenzaldehyde (1, 2 g, 14.48 mmol) in dichloromethane (40 mL). After 15 minutes, the solution became clear and it was cooled down to 0° C. and TBSCl was added as the silylating agent (5.019 g, 33.30 mmol). The reaction mixture was brought to room temperature and stirred for 14 hours. Subsequently, the mixture was poured to 200 mL cold water and extracted with dichloromethane (3×100 mL). Combined organic layers were washed with brine, dried with anhydrous MgSO₄ and the solvent was evaporated. The product was purified with column chromatography on silica gel using EtOAc: hexane (1:10) system as the eluent. Compound 2 in the form of white solid was obtained (5.033 g, 95%).

In the aforementioned reaction, the silylating agent is added at about 0° C. However, the further reaction is carried out at a temperature higher than 0° C. but not higher than room temperature.

TBSCl was used as the silylating agent in this reaction. However, other alkylsilyl halides commonly used for protecting the hydroxy group can be also used in this reaction.

Furthermore, the imidazole used as the activating agent for the silylating agent can be replaced with other reagents, such as a mixture of triethylamine with 4-dimethylaminopyridine, triethylamine with 1,8-diazabicyclo[5.4.0]undec-7-ene or 18-crown-6 ether with potassium hydride. Furthermore, other organic aprotic solvents such as dimethylformamide, dichloromethane, acetonitrile, tetrahydrofuran or toluene, can be also used as the solvent.

The reaction is carried out for 4 to 24 hours while monitoring the progress of the reaction using thin-layer chromatography (TLC). The reaction time specified in the example corresponds to the time point after which no changes noticeable on the TCL plate have occurred in the system.

Step b): The strong base (n-BuLi, 1.6 M in THF, 10.306 mL, 16.5 mmol) was added to the suspension of CH₃PPh₃Br (5.890 g, 16.5 mmol) in anhydrous THF (18 mL) at −78° C. After 20 minutes, solution of aldehyde 2 (5.033 g, 13.75 mmol) in anhydrous THF (9.5 mL) was added using a cannula. The reaction mixture was brought to room temperature and stirred for another 4 hours. The reaction was quenched by adding 30 mL brine. It was extracted with CH₂Cl₂ (3×50 mL). Combined organic layers were washed with water, dried with anhydrous MgSO₄ and the solvent was evaporated. The product was purified with column chromatography on silica gel using Et₂O:hexane (1:100) system as the eluent. Compound 3 in the form of colorless oil was obtained (4.611 g, 92%).

The reagents in the aforementioned reaction are added at the temperature of about −78° C.

However, the further reaction can be carried out at a temperature in the range from −78° C. to room temperature.

Any alkyltriphenylphosphonium halide commonly employed in reactions for converting aldehyde group to alkene group can be used in the aforementioned reaction. In addition, other strong bases, such as lithium diisopropylamide, potassium tert-butoxide or potassium bis(trimethylsilyl)amide can be used instead of n-BuLi. Furthermore, toluene and dichloromethane are also suitable as the solvent.

Stirring of the reaction mixture at room temperature will be extended to 24 hours if analysis using TLC chromatography shows that the reaction has not yet been completed.

Step c): In a dry vial containing a magnetic stirrer bar, compound 3 (1 g, 2.73 mmol), 4-iodobenzoic acid ethyl ester (0.73 g, 2.60 mmol), Pd(OAc)₂ catalyst (0.031 g, 0.14 mmol) and the tri(o-tolyl)phosphine (ToP) ligand (0.063 g, 0.21 mmol) were placed in anhydrous triethylamine (4.5 mL) under argon atmosphere. The mixture in the sealed vial was stirred for 2 hours at 60° C., and subsequently for 24 hours at 80° C. After cooling down, the mixture was diluted by adding CH₂Cl₂ and poured into water. The aqueous layer was extracted with dichloromethane (3×20 mL). Combined organic layers were washed with brine, dried with anhydrous MgSO₄ and the solvent was evaporated. The product was purified with column chromatography on silica gel using Et₂O:hexane (1:9) system as the eluent. Compound 4 in the form of white solid was obtained (0.97 g, 71%).

In the above reaction, the type of halogenated benzene derivative substituted with at least one ester group of formula —(CH₂)ₓ—C(O)OR, wherein R is a C₁-C₃ alkyl group, and x is an integer of 0 to 2, depends on how many OH groups are to be substituted in the final polyphenol derivative with the groups containing the nitroxyl radical and on the type of linking group that connects the group containing the nitroxyl radical with the polyphenol molecule. If the final derivative is to contain one group containing the nitroxyl radical, the benzene derivative is a derivative containing one ester group, and if the final derivative is to contain two groups containing the nitroxyl radical, the benzene derivative is a derivative containing two ester groups (Example 2a below). In addition, the ester group can be —C(O)OR, —CH₂C(O)OR or —(CH₂)₂—C(O)OR group, wherein R is methyl, ethyl or propyl. Generally, reaction conditions are identical irrespective of the type of the halogenated benzene derivative being used.

In the aforementioned reaction, other palladium (0) or (II) complexes can be used as the catalyst and other phosphonium ligands commonly employed in the coupling reaction between an alkene group and a benzene derivative can be used as the ligand.

Triethylamine is used as the base in the reaction. Triethylamine is also the solvent in this case. However, the reaction can be also carried out in other organic aprotic solvents, such as dichloromethane, dimethylformamide, toluene, tetrahydrofuran, acetonitrile or ether.

In the aforementioned reaction, the temperature in the range from 50° C. to 90° C. was preferably used, that is, initially 60° C. and subsequently 80° C. However, according to the generally available knowledge of chemistry, palladium-catalyzed coupling reactions can be carried out at a temperature in the range from room temperature to 190° C.

Step d): The solution of compound 4 (2 g, 4.0 mmol) in anhydrous CH₂Cl₂ (60 mL) was cooled down to −78° C., and DIBALH was added dropwise as the reducing agent (1 M in CH₂Cl₂, 12 mL, 12.0 mmol) and stirred at that temperature for 2.5 hours. The reaction was quenched by adding sodium potassium tartrate solution (1 M, 3 mL), HCl (1 M, 3 mL) and water (12 mL). The mixture was left for 2 hours (to reach room temperature). Subsequently, the mixture was poured to 30 mL water and the layers were separated. The aqueous layer was extracted with dichloromethane (3×60 mL). Combined organic layers were washed with brine, dried with anhydrous MgSO₄ and the solvent was evaporated. The product was purified with column chromatography on silica gel using EtOAc:hexane (1:4) system as the eluent. Compound 5 in the form of white solid was obtained (1.62 g, 86%).

In the above reaction, identical results are obtained when lithium aluminum hydride is used as the reducing agent. In addition, the reaction can be also carried out in other aprotic solvents, such as dichloromethane, dimethylformamide, toluene, tetrahydrofuran, acetonitrile or ether.

The reaction is carried out for 1 to 24 hours while monitoring the progress of the reaction using thin-layer chromatography (TLC). The reaction time specified in the example corresponds to the time point after which no changes noticeable on the TCL plate have occurred in the system.

The aforementioned reaction is preferably carried out at a temperature selected from the range from −78° C. to −50° C., because the reaction between DIBALH and compound 4 proceeds quickly and there is no need to use higher temperatures. However, according to the generally available knowledge of chemistry, the reduction reaction of ester group to alcohol group can be carried out at a temperature in the range from −78° C. to room temperature. The prerequisite is to add the reducing agent at reduced temperature considering its high reactivity.

Step e): Compound 5 (1.62 g, 3.4 mmol) was dissolved in CH₂Cl₂ (18 mL) and PDC was added as the oxidizing agent (3.20 g, 8.5 mmol) in two equal portions. The reaction was carried out at room temperature for 6 hours. Subsequently, it was filtered through a Celite layer and the filtrate was concentrated using an evaporator. The product was purified with column chromatography on silica gel using EtOAc:hexane (5:95) system as the eluent. Compound 6 in the form of white solid was obtained (1.484 g, 92%).

In the above reaction, pyridinium dichromate (PDC) was used as the oxidizing agent; however, in this reaction, other oxidizing agents commonly used for the oxidation of alcohol group to aldehyde group can be used, such as pyridinium chlorochromate, oxalyl chloride, triethylamine in dichloromethane, tetrapropylammonium perruthenate or 4-methylmorpholine 4-oxide in tetrahydrofuran. Furthermore, other aprotic solvents known in the art can be used as the solvent, such as dichloromethane, dimethylformamide, toluene, tetrahydrofuran, acetonitrile or ether.

The reaction can be also carried out at the temperature close to 0° C. However, this involves the reaction time being extended to about 18 hours and, therefore, the reaction is preferably carried out at a temperature in the range from 10° C. to room temperature.

Step f): Compound 6 (1.484 g, 3.17 mmol) was dissolved in the mixture of t-BuOH (30 mL) and THF (9 mL) at room temperature. Subsequently, the solution was cooled down to 0° C. and 2-methylbut-2-ene (5.19 mL, 49.26 mmol) was added. After 20 minutes, solution of NaH₂PO₄×H₂O (3.782 g, 9.57 mmol) and NaClO₂ (80%, 0.863 g, 7.66 mol) in H₂O (16 mL) was added dropwise as the oxidizing agent. The reaction mixture was stirred for 5 hours at 0-3° C. and quenched by adding solid Na₂SO₃ (1.23 g, 9.75 mmol). The aqueous layer was extracted with EtOAc (4×100 mL), dried with anhydrous MgSO₄ and the solvent was evaporated. The product was purified with column chromatography on silica gel using EtOAc:hexane (1:1) system as the eluent. Compound 7 in the form of white solid was obtained (0.967 g, 63%).

The reagents in the aforementioned reaction are added at the temperature of about 0° C. However, the further reaction can be also carried out at room temperature, but it is preferably carried out in the range from 0° C. to 3° C.

Step g): Compound 7 (0.967 g, 1.99 mmol) was dissolved in CH₂Cl₂ (35 mL), and EDCI as the activating agent for carboxy group (0.770 g, 4.97 mmol), DMAP as the deprotonating agent for hydroxy group (0.243 g, 1.99 mmol) and 4-hydroxy-TEMPO (0.515 g, 2.99 mmol) were subsequently added. The reaction was carried out at room temperature for 16 hours and quenched by adding 20 mL of 10% sodium carbonate solution. Layers were separated and the aqueous layer was extracted with EtOAc (3×20 mL). Combined organic layers were washed with H₂O, dried with anhydrous MgSO₄ and the solvent was evaporated. The product was purified with column chromatography on silica gel using MeOH:CH₂Cl₂ (5:95) system as the eluent. Compound 8 in the form of brown solid was obtained (1.096 g, 86%).

DDC can be also used as the activating agent for carboxy group.

In the aforementioned synthesis, 4-hydroxy-2,2,6,6-tetramethylpiperidine-1-oxyl compound was used as the heterocyclic compound containing one nitrogen atom in the form of nitroxyl radical; however, other compounds containing the nitroxyl radical, being derivatives of piperidine, pyridine, pyrrolidine, pyrroline and pyrrole, in which both carbon atoms adjacent to the nitrogen atom in the form of the nitroxyl radical are substituted independently of each other with one or two (identical or different) methyl, ethyl or propyl groups, and in which one of the other carbon atoms is substituted with hydroxy group, can be also used in the identical conditions.

The reaction can be also carried out in other organic aprotic solvents, such as dichloromethane, dimethylformamide, toluene, tetrahydrofuran, acetonitrile or ether.

Slightly higher temperatures, i.e. up to 30° C., can be also used with similar results in this reaction.

Step h): Compound 8 (1.096 g, 1.71 mmol) was dissolved in anhydrous THF (50 mL), cooled down to 0° C., and TBAF was subsequently added as the source of fluoride ions (1 M in THF, 4.27 mL, 4.27 mmol). The reaction mixture was heated to room temperature, the mixture was stirred for 2 hours and it was quenched by adding 20 mL brine. It was extracted with EtOAc (3×40 mL). Combined organic layers were washed with H₂O, dried with anhydrous MgSO₄ and the solvent was evaporated. The product was purified with column chromatography on silica gel using MeOH:CH₂Cl₂ (1:9) system as the eluent. Brown solid was obtained which was subsequently recrystallized from Et₂O. Compound H3 in the form of beige solid was obtained (0.549 g, 78%).

The reaction can be also carried out in other organic aprotic solvents, such as dichloromethane, dimethylformamide, toluene, tetrahydrofuran, acetonitrile or ether.

In the aforementioned reaction, a temperature in the range from 0 to 5° C. is preferably used when adding the reagent, and the mixture is subsequently left to reach room temperature and is stirred at that temperature for appropriate time.
b) The synthesis of nitroxyl resveratrol derivative containing one free hydroxy group:

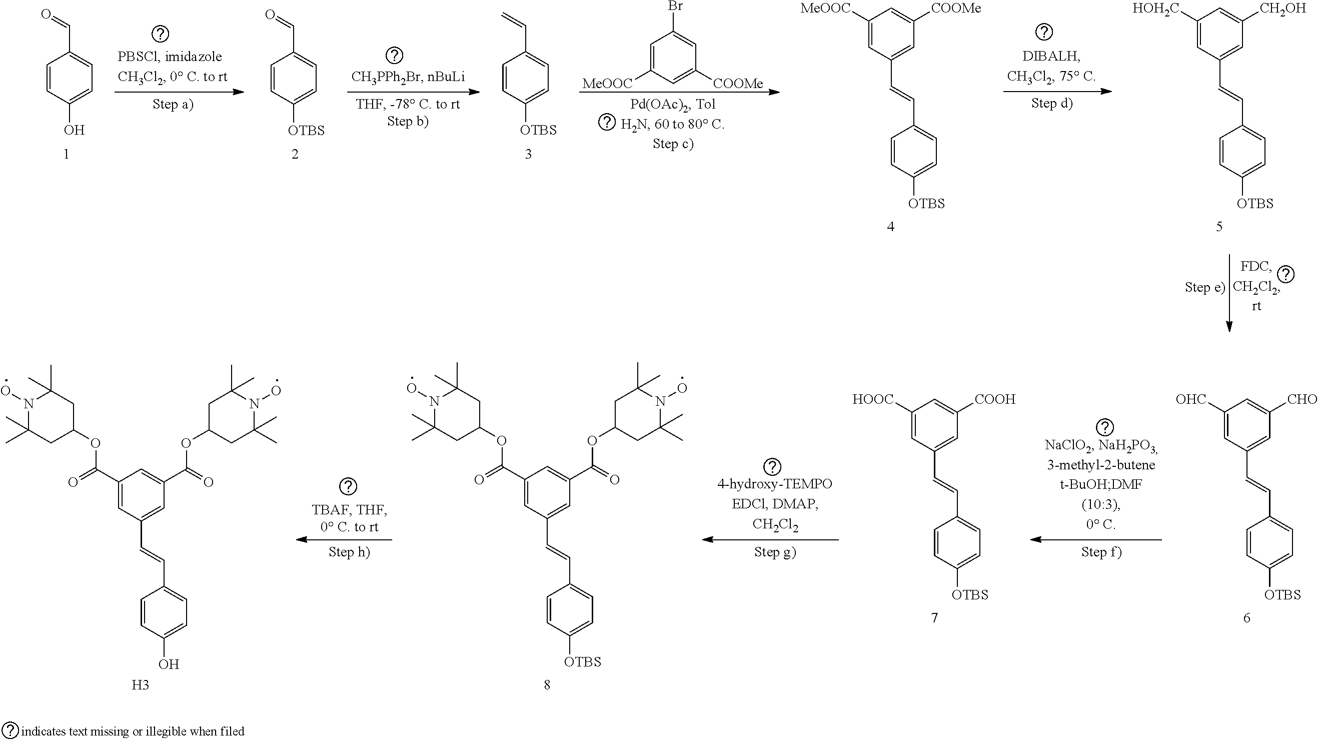

Step a): Imidazole as the activating agent for the silylating agent (3.35 g, 49.14 mmol) was added to the stirred suspension of 4-hydroxybenzaldehyde (1, 2 g, 16.38 mmol) in dichloromethane (40 mL) and cooled down to 0° C. TBSCl (2.96 g, 19.66 mmol) was added. The mixture was brought to room temperature and stirred for 5 hours and was subsequently poured to 200 mL cold water and extracted with dichloromethane (3×50 mL). Combined organic layers were washed with brine, dried with anhydrous MgSO₄ and the solvent was evaporated. The product was purified with column chromatography on silica gel using EtOAc:hexane (1:10) system as the eluent. Compound 2 in the form of white solid was obtained (3.560 g, 92%).

Step b): n-BuLi (1.6 M in THF, 11.31 mL, 18.10 mmol) was added to the suspension of CH₃PPh₃Br (6.461 g, 16.5 mmol) in anhydrous THF (19 mL) at −78° C. After 20 minutes, solution of aldehyde 2 (3.560 g, 15.08 mmol) in anhydrous THF (10 mL) was added using a cannula. The mixture was brought to room temperature over 1 hour and stirred at that temperature for further 4 hours. The reaction was quenched by adding 30 mL brine. It was extracted with CH₂Cl₂ (3×40 mL). Combined organic layers were washed with water, dried with anhydrous MgSO₄ and the solvent was evaporated. The product was purified with column chromatography on silica gel using Et₂O:hexane (1:100) system as the eluent. Compound 3 in the form of colorless oil was obtained (3.142 g, 89%).

Step c): In a dry vial, compound 3 (2.0 g, 8.545 mmol), dimethyl 5-bromoisophthalate (2.123 g, 7.768 mmol), Pd(OAc)₂ (0.096 g, 0.427 mmol), and tri(o-tolyl)phosphine (0.196 g, 0.644 mmol) were placed in anhydrous triethylamine (15 mL) under argon atmosphere. The mixture in the sealed vial was stirred for 2 hours at 60° C., and subsequently for 38 hours at 80° C. After cooling down, the mixture was diluted in CH₂Cl₂ and poured into water. The aqueous layer was extracted with dichloromethane (3×40 mL). Combined organic layers were washed with brine, dried with anhydrous MgSO₄ and the solvent was evaporated. The product was purified with column chromatography on silica gel using Et₂O:hexane (1:9) system as the eluent. Compound 4 in the form of white solid was obtained (2.220 g, 67%).

Step d): The solution of compound 4 (2.22 g, 5.21 mmol) in anhydrous CH₂Cl₂ (78 mL) was cooled down to −78° C., DIBALH was added dropwise (1 M in CH₂Cl₂, 26.05 mL, 26.05 mmol) and the mixture was stirred at that temperature for 2.5 hours. The reaction was quenched by adding sodium potassium tartrate solution (1 M, 6 mL), HCl (1 M, 6 mL) and water (24 mL), and after bringing to room temperature, the mixture was stirred for 2 hours. Subsequently, the mixture was poured to 50 mL water and the layers were separated. The aqueous layer was extracted with dichloromethane (3×100 mL). Combined organic layers were washed with brine, dried with anhydrous MgSO₄ and the solvent was evaporated. The product was purified with column chromatography on silica gel using EtOAc:hexane (1:1) system as the eluent. Compound 5 in the form of white solid was obtained (1.72 g, 89%).

Step e): Compound 5 (1.72 g, 4.6 mmol) was dissolved in CH₂Cl₂ (24 mL) and PDC (8.66 g, 23 mmol) was added in two equal portions. The reaction was carried out at room temperature for 16 hours. Subsequently, the mixture was filtered through a Celite layer and the filtrate was concentrated using an evaporator. The product was purified with column chromatography on silica gel using EtOAc:hexane (1:4) system as the eluent. Compound 6 in the form of white solid was obtained (1.395 g, 82%).

Step f): Compound 6 (1.395 g, 3.81 mmol) was dissolved in the mixture of t-BuOH (40 mL) and THF (12 mL) at room temperature. Subsequently, the solution was cooled down to 0° C. and 2-methylbut-2-ene (6.24 mL, 59.21 mmol) was added. After 20 minutes, solution of NaH₂PO₄×H₂O (9.03 g, 22.86 mmol) and NaClO₂ (80%, 2.06 g, 18.30 mol) in H₂O (38 mL) was added dropwise. The reaction mixture was stirred for 5 hours at 0-3° C. and quenched by adding solid Na₂SO₃ (2.94 g, 23.29 mmol). The aqueous layer was extracted with EtOAc (8×60 mL), dried with anhydrous MgSO₄ and the solvent was evaporated. The product was purified with column chromatography on silica gel using MeOH:CH₂Cl₂ (from 1:1 to 3:2) with 0.1% AcCOOH as the eluent. Compound 7 in the form of white solid was obtained (0.896 g, 59%).

Step g): Compound 7 (0.896 g, 2.25 mmol) was dissolved in CH₂Cl₂ (39 mL), and subsequently EDCI (1.44 g, 9.29 mmol), DMAP (0.824 g, 6.75 mola) and 4-hydroxy-TEMPO (1.35 g, 7.87 mol) were added. The reaction was carried out at room temperature for 52 hours and quenched by adding 20 mL of 10% sodium carbonate solution. Layers were separated and the aqueous layer was extracted with EtOAc (3×20 mL). Combined organic layers were washed with H₂O, dried with anhydrous MgSO₄ and the solvent was evaporated. The product was purified with column chromatography using EtOAc:hexane (1:4) as the eluent. Compound 8 in the form of orange solid was obtained (1.176 g, 74%).

Step h): Compound 8 (1.76 g, 1.83 mmol) was dissolved in anhydrous THF (52 mL), cooled down to 0° C., and TBAF was subsequently added (1 M in THF, 2.75 mL, 2.75 mmol). The reaction mixture was heated to room temperature, stirred for 2 hours and it was quenched by adding 20 mL brine. The mixture was extracted with EtOAc (3×40 mL). Combined organic layers were washed with H₂O, dried with anhydrous MgSO₄ and the solvent was evaporated. The product was purified with column chromatography using EtOAc:hexane (2:3) system as the eluent. Orange solid was obtained which was subsequently recrystallized from Et₂O. Compound H2 in the form of beige solid was obtained (0.783 g, 80%).

### Example 3: Antioxidant/Antiaging Properties Tests

Three lines of human skin fibroblasts obtained from healthy volunteers were used in cell-based tests to investigate the effect of biological variation on the effectiveness of action of the tested compounds. The cells were subcultured and seeded onto a 6- or 12-well plate (cell density: 5,000/cm² of area). The cells were placed in the incubator in conditions optimal for proliferation (37° C., 5% CO₂) for 24 hours. After a required time has elapsed, hydrogen peroxide (concentration: 200 μM, experimentally determined as optimal for inducing fibroblast aging) and the tested compounds H3 and H5; or the tested compounds alone were added to the cells to determine the cytotoxicity of the compounds. Compound H3 was the resveratrol derivative with one nitroxyl group, and compound H5 was the nitroxyl curcumin derivative. The cells were subsequently placed in the incubator for 24 hours, 48 hours, 72 hours or 7 days depending on the type of the experiment being conducted. Subsequently, the number of cells in the culture was counted, and the percentage of proliferating cells or the percentage of SA-β-gal-positive cells was determined.

FIG. 1 shows the number of cells in the culture after treatment with substances H3 and H5. The horizontal line in the chart indicates the treatment point, and the results were normalized to this number. As can be seen from the data obtained, both prepared compounds have no cytotoxic properties with respect to human skin fibroblasts at a concentration of up to 5 μM (the experimentally determined value; the active concentrations of the tested compounds are shown in the Figures).

The next step was to determine cell proliferation based on a bromodeoxyuridine (BrdU) incorporation test. Being a synthetic nucleoside (thymidine) analog, BrdU is incorporated in the DNA molecule in the S stage of cell division. As a result of immunocytochemistry staining of cell nuclei with BrdU (FIG. 2), information about the % of cultured cells that undergo division, is obtained. The tests confirmed that compounds H3 and H5 do not have any cytotoxic properties (the percentage of BrdU-positive cells was similar to that in control).

Subsequently, the activity of senescence-associated-β-galactosidase (SA-β-gal) was tested. Increased activity of SA-β-gal is observed in the cells that underwent senescence (one of the markers of aging). FIG. 3 shows the results which demonstrate that the % of cells with increased SA-β-gal activity after treatment with H3 and H5 is similar to untreated control.

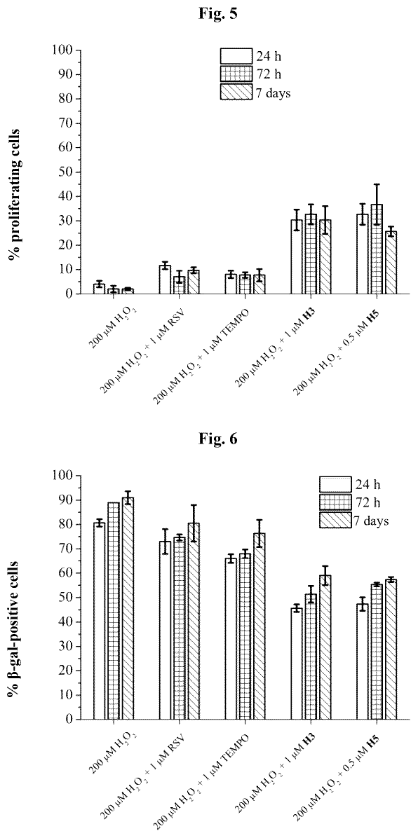

The next step was to investigate whether the prepared compounds protect cells against aging induced by oxidative stress. For this, 200 μM hydrogen peroxide was used (the concentration was determined experimentally). Different concentrations of H3 and H5 were added to the cell cultures in which accelerated aging was induced. The results show that the compounds protect cells against the consequences of oxidative stress (their number increases compared to the culture to which only hydrogen peroxide was added, FIG. 4). The compounds showed a protective effect and the % of dividing cells was 10-fold higher compared to cells treated with H₂O₂ alone (40% vs. 4%; FIG. 5). When added to the cells treated with hydrogen peroxide, H3 and H5 reduced the number of cells with increased SA-β-gal activity from 90% (H₂O₂) to 60% (H₂O₂+H3 or H5), as shown in FIG. 6. It is also worth emphasizing that it has been shown that the obtained compound H3 has much better activity than the components from which it is built (resveratrol (RSV) and TEMPO).

Based on the above results, it can be assumed that derivatives of other polyphenols, such as quercetin, genistein and daidzein, after substituting their hydroxyl groups with groups containing the nitroxyl radical as discussed above, will show antioxidant/antiaging properties similar to those of the obtained derivatives, due to the presence of similar main groups in the molecule that are responsible for these properties.

1. A nitroxyl polyphenol derivative of formula: 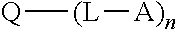
wherein Q is a group derived from
a polyphenol being curcumin, quercetin, genistein or daidzein, and L is an ester linking group of formula —OC(O)—(CH₂)ₓ— wherein x is an integer of 0 to 2; or
a polyphenol being resveratrol, and L is an ester linking group of formula —(CH₂)ₓ—C(O)O—, wherein x is an integer of 0 to 2; and A is a 5- or 6-membered heterocyclic group containing one nitrogen atom which is in the form of nitroxyl radical (NO·), in which both carbon atoms adjacent to the nitroxyl radical are substituted independently of each other with one or two C₁-C₃ alkyl groups; and
n is an integer of 1 to 5.

2. (canceled)

3. The nitroxyl polyphenol derivative according to claim 1, wherein Q is a group derived from a polyphenol being curcumin, and L is an ester linking group of formula —OC(O)—(CH₂)ₓ—, wherein x is an integer of 0 to 2.

4-6. (canceled)

7. The nitroxyl polyphenol derivative according to claim 1, wherein A is a piperidine-1-oxyl group in which both carbon atoms adjacent to the nitroxyl radical are substituted independently of each other with two C₁-C₃ alkyl groups, preferably 2,2,6,6-tetramethylpiperidine-1-oxyl group.

8. (canceled)

9. The nitroxyl polyphenol derivative according to claim 1, wherein n is 1 or 2.

10. The nitroxyl polyphenol derivative according to claim 1, which is the derivative selected from the following:

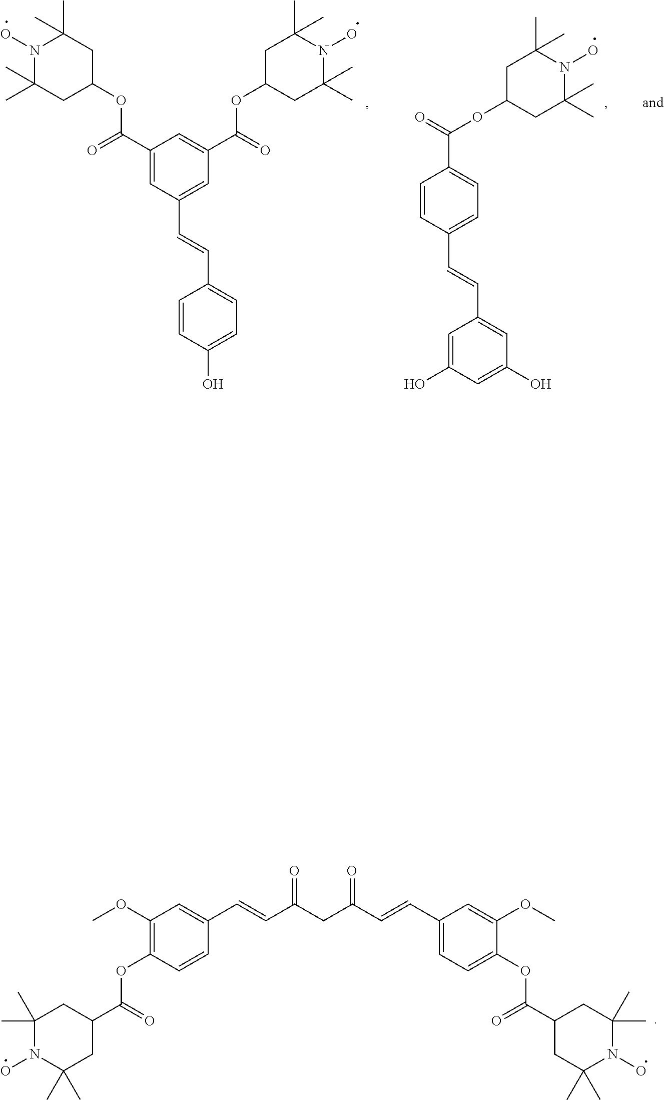

11-12. (canceled)

13. A method for the preparation of nitroxyl polyphenol derivative of formula: 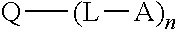
wherein Q is a group derived from a polyphenol being curcumin, quercetin, genistein or daidzein;
L is an ester linking group of formula —OC(O)—(CH₂)ₓ—, wherein x is an integer of 0 to 2;
A is a 5- or 6-membered heterocyclic group containing one nitrogen atom which is in the form of nitroxyl radical (NO), in which both carbon atoms adjacent to the nitroxyl radical are substituted independently of each other with one or two C₁-C₃ alkyl groups; and
n is an integer of 1 to 5;
characterized in that the method comprises a reaction of curcumin, quercetin, genistein or daidzein with a 5- or 6-membered heterocyclic compound containing one nitrogen atom which is in the form of nitroxyl radical (NO), in which both carbon atoms adjacent to the nitroxyl radical are substituted independently of each other with one or two C₁-C₃ alkyl groups and in which one of the other carbon atoms is substituted with a carboxy group of general formula —(CH₂)ₓ—C(O)OH, wherein x is an integer of 0 to 2;
wherein the reaction is carried out in an organic solvent at a temperature in the range from −10° C. to a temperature lower than room temperature, in the presence of a deprotonating agent for hydroxy group and an activating agent for carboxy group.

14. (canceled)

15. The method according to claim 13, characterized in that a piperidine-1-oxyl compound, in which both carbon atoms adjacent to the nitroxyl radical are substituted independently of each other with two C₁-C₃ alkyl groups and in which one of the other carbon atoms is substituted with a carboxy group of general formula —(CH₂)ₓ—C(O)OH, wherein x is an integer of 0 to 2, preferably 4-carboxy-2,2,6,6-tetramethylpiperidine-1-oxyl compound, is used as the 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with a carboxy group of general formula —(CH₂)ₓ—C(O)OH, wherein x is an integer of 0 to 2.

16. (canceled)

17. The method according to claim 13, characterized in that 4-dimethylaminopyridine is used as the deprotonating agent for hydroxy group; and/or
1-ethyl-3-(3-dimethylaminopropyl)carbodiimide or N,N′-dicyclohexylcarbodiimide, preferably 1-ethyl-3-(3-dimethylaminopropyl)carbodiimide, is used as the activating agent for carboxy group; and/or
an aprotic polar solvent, preferably dichloromethane, is used as the organic solvent; and/or
the reaction is carried out at a temperature in the range from −10° C. to 0° C.

18-27. (canceled)

28. A method for the preparation of nitroxyl polyphenol derivative of formula: 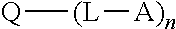
wherein:
Q is a group derived from a polyphenol being resveratrol;
L is an ester linking group of formula —(CH₂)ₓ—C(O)O—, wherein x is an integer of 0 to 2;
A is a 5- or 6-membered heterocyclic group containing one nitrogen atom which is in the form of nitroxyl radical (NO·), in which both carbon atoms adjacent to the nitroxyl radical are substituted independently of each other with one or two C₁-C₃ alkyl groups; and
n is an integer of 1 or 2;
characterized in that the method comprises the following steps:
a) a reaction between a benzaldehyde derivative substituted with at least one hydroxy group and a silylating agent to protect at least one hydroxy group, wherein the reaction is carried out at a temperature in the range from 0° C. to room temperature in an organic solvent in the presence of an activating agent for the silylating agent;
b) a reaction of the protected benzaldehyde derivative prepared in step a) with an alkyltriphenylphosphonium halide to convert aldehyde group to alkene group, wherein the reaction is carried out in an organic solvent at a temperature in the range from −78° C. to room temperature, in the presence of a strong base;
c) a coupling reaction of alkene group in the derivative prepared in step b) with a halogenated benzene derivative substituted with at least one ester group of formula —(CH₂)ₓ—C(O)OR, wherein R is a C₁-C₃ alkyl group, and x is an integer of 0 to 2; wherein the reaction is carried out in the presence of a catalyst, a phosphonium ligand and a base, optionally in an organic solvent, at a temperature in the range from room temperature to 190° C.;
d) a reaction of the derivative prepared in step c) with a reducing agent to reduce at least one ester group to at least one alcohol group, wherein the reaction is carried out in an organic solvent at a temperature in the range from −78° C. to room temperature;
e) a reaction of the derivative prepared in step d) with an oxidizing agent to oxidize at least one alcohol group to at least one aldehyde group, wherein the reaction is carried out in an organic solvent at a temperature in the range from 0° C. to room temperature;
f) a reaction of the derivative prepared in step e) with an oxidizing agent to oxidize at least one aldehyde group to at least one acid group, wherein the reaction is carried out in an organic solvent or a mixture of organic solvents at a temperature in the range from 0° C. to room temperature;
g) a reaction of the derivative prepared in step f) with a 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with hydroxy group, to prepare a nitroxyl polyphenol derivative containing at least one protected hydroxy group, wherein the reaction is carried out in an organic solvent at a temperature in the range from room temperature to 30° C., in the presence of a deprotonating agent for hydroxy group and an activating agent for carboxy group; and
h) a reaction of the nitroxyl polyphenol derivative containing at least one protected hydroxy group prepared in step g) with a reagent serving as a source of fluoride ions to deprotect at least one hydroxy group, wherein the reaction is carried out in an organic solvent at a temperature in the range from 0° C. to room temperature.

29. The method according to of claim 28, characterized in that, in step a), hydroxy- or dihydroxybenzaldehyde is used as the benzaldehyde derivative, and/or an alkylsilyl halide, preferably tert-butyldimethylsilyl chloride is used as the silylating agent, and/or imidazole, a mixture of triethylamine with 4-dimethylaminopyridine or with 1,8-diazabicyclo[5.4.0]undec-7-ene or a mixture of 18-crown-6 ether with potassium hydride, preferably imidazole alone is used as the activating agent for the silylating agent.

30-34. (canceled)

35. The method according to claim 28, characterized in that, in step b), methyltriphenylphosphonium bromide is used as the alkyltriphenylphosphonium halide, and/or n-butyllithium, lithium diisopropylamide, potassium tert-butoxide or potassium bis(trimethylsilyl)amide, preferably n-butyllithium is used as the strong base.

36-43. (canceled)

44. The method according to claim 28, characterized in that the base being used is the organic solvent in step c); and/or
characterized in that the reaction in step c) is carried out at a temperature in the range from 50 to 90° C.; and/or
characterized in that, in step c), 4-iodobenzoic acid ethyl ester or dimethyl 5-bromoisophthalate is used as the halogenated benzene derivative substituted with at least one ester group of formula —(CH₂)ₓ—C(O) OR, wherein R is a C₁-C₃ alkyl group, and x is an integer of 0 to 2, and/or a palladium (0) or (II) complex, preferably palladium acetate is used as the catalyst and/or tri(o-tolyl)phosphine is used as the phosphonium ligand, and/or triethylamine is used as the base.

45. The method according to claim 28, characterized in that diisobutylaluminum hydride or lithium aluminum hydride, preferably diisobutylaluminum hydride is used as the reducing agent in step d); and/or
characterized in that the reaction in step d) is carried out at a temperature in the range from −78° C. to −50° C.

46-50. (canceled)

51. The method according to claim 28, characterized in that pyridinium dichromate, pyridinium chlorochromate, oxalyl chloride, triethylamine in dichloromethane, tetrapropylammonium perruthenate or 4-methylmorpholine 4-oxide in tetrahydrofuran, preferably pyridinium dichromate is used as the oxidizing agent in step e); and/or
characterized in that the reaction in step e) is carried out at a temperature in the range from 10° C. to room temperature.

52-54. (canceled)

55. The method according to claim 28, characterized in that, in step f), NaClO₂ and NaH₂PO₄·H₂O solution is used as the oxidizing agent and/or tert-butanol, tetrahydrofuran or 2-methylbut-2-ene or a mixture thereof is used as the organic solvent; and/or
characterized in that the reaction in step f) is carried out at a temperature in the range from 0 to 3° C.

56-58. (canceled)

59. The method according to claim 28, characterized in that, in step g), a hydroxypiperidine-1-oxyl compound, in which both carbon atoms adjacent to the nitroxyl radical are substituted independently of each other with two C₁-C₃ alkyl groups, preferably 4-hydroxy-2,2,6,6-tetramethylpiperidine-1-oxyl compound, is used as the 5- or 6-membered heterocyclic compound containing one nitrogen atom which is in the form of nitroxyl radical (NO·), in which both carbon atoms adjacent to the nitroxyl radical are substituted independently of each other with one or two C₁-C₃ alkyl groups and in which one of the other carbon atoms is substituted with hydroxy group, and/or 4-dimethylaminopyridine is used as the deprotonating agent for hydroxy group, and/or 1-ethyl-3-(3-dimethylaminopropyl)carbodiimide or N,N′-dicyclohexylcarbodiimide, preferably 1-ethyl-3-(3-dimethylaminopropyl)carbodiimide, is used as the activating agent for carboxy group.

60-64. (canceled)

65. The method according to claim 28, characterized in that tetra-n-butylamine fluoride is used as the reagent serving as a source of fluoride ions in step h).

66. (canceled)

67. The method according to claim 28, characterized in that in steps from a) to e) and g) and h) aprotic solvent, preferably dichloromethane, dimethylformamide, toluene, tetrahydrofuran, acetonitrile or ether is used as the organic solvent.

68. (canceled)

69. A method of inhibiting oxidation of a cell, comprising administering the nitroxyl polyphenol derivative as defined in claim 1 as an antioxidant agent to the cell.

70. A method of delaying aging of a cell, comprising administering the nitroxyl polyphenol derivative as defined in claim 1 as an antiaging agent to the cell.
